(19) United States
(12) Patent Application Publication
(10) Pub. No.: US 2010/0318007 A1
(43) Pub. Date: Dec. 16, 2010

(54) ELECTROMECHANICAL TACTILE STIMULATION DEVICES AND METHODS

(76) Inventor: DONALD J. O'BRIEN, Huntington Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/482,269

(22) Filed: Jun. 10, 2009

(51) Int. Cl. A61H 1/00; A61M 21/00; A61F 5/56

(52) U.S. Cl. 601/48; 600/27; 128/848

(57) ABSTRACT

A device for stimulating a sleeper is disclosed. The device comprises a tactile electromechanical stimulator positioned against the sleeper's back proximate the sleeper's lower spine. The stimulator is actuated by a control circuit and powered by a power source. In one embodiment, the control circuit can be programmed to occasionally actuate the stimulator to promote more restful sleep. In one embodiment, the control circuit includes a receiver, and a transmitter is included that is adapted to transmit an actuation signal upon detection of excess noise or motion from the sleeper. The receiver, upon receiving the actuation signal, can actuate the stimulator for a randomized period of time via one or more randomized output pulses generated by a controller in communication with the receiver.

### BACKGROUND

1. Field

Embodiments generally relate to sleep enhancement and more particularly to electromechanical tactile stimulation devices and methods for promoting restful sleep.

2. Description of the Related Art

Tens of millions of sleepers, including children, have undesirable sleep disorders that prevent restful sleep. One prevalent disorder is loud snoring, but there are many others, called parasomnias, that cause disruptions of sleep. These parasomnias include: bruxism, or involuntary grinding of teeth while sleeping; somnambulism, or sleep walking; Restless Legs Syndrome; and insomnia, the inability to fall asleep or remain asleep for a reasonable amount of time. Sleep disorders are symptomatic of sleep that is not restful, either for the sleeper or those sleeping nearby. While there have been electrically based attempts to solve the disorder of snoring, up to the present time a satisfactory solution has not been found to suppress other sleep disorders with the same apparatus.

Behavior modification by electrically operated devices external to the body was originally affected by an electric shock. The subject of this type of aversive treatment can be deprived of a restful sleep. Other electronic devices depend on audible or vibration alarms to avert the undesirable sonic behavior of a sleeper. These devices rely on causing the offending sleeper to awaken and require resetting of an alarm or changing sleeping position and thus such devices are only a continuation of the disturbing effect on the sleeper's restfulness. Some of the devices display a record of the total number of times aversive stimulus was required to attempt to improve the user's sleep, however mildly.

The above electronic devices are designed to modify the audible sleeping behavior of a person by training the sleeper via aversive stimulus to repress the sounds. In the process, these devices can disrupt the actual purpose of sleeping: an uninterrupted, restful recuperation from the previous day's accumulation of physical and emotional stresses.

In addition, conventional anti-snoring devices may employ a stimulus that is unchanged during each necessary application and which may therefore become monotonous. This is a disadvantage due to the well-known ability of the human central nervous system to accommodate and ignore repetitive stimulation.

Devices that claim to treat the condition of improper muscle tone have been invented to treat the breathing problems involved in sleep apnea. These devices attempt to improve the breathing of a sleeper by focusing on the tone of the upper airway muscles of the throat with devices that intrude on a sleeper's ability to get a restful sleep.

Current solutions for snoring and other sleep disorders including drug treatments, oral appliances, implanted devices, and surgery make no attempt to induce uniform muscle tone throughout the body. Any attempts to treat disorders of muscle tone involving breathing problems focus on local treatment of the upper airway passages of the throat with complicated electrical sensing and stimulation. Although it is thoroughly documented in medical texts that snoring is instigated by the excessive or improper relaxation of the muscles controlling the pharyngeal tissues of the upper airway passages, current solutions for snoring focus on the symptoms rather than the cause, which is believed to be instigated by a lack of uniform muscle tone throughout the entire body including the throat area. Inducing and maintaining proper over-all muscle tone while sleeping suppresses sleep disorders resulting from this anomaly.

### SUMMARY

In view of the foregoing, a need exists for electromechanical tactile stimulation devices and methods that enhance sleep without disrupting a person's sleep. In certain embodiments, such devices and methods can be used to improve uniform muscle tone throughout the body. In other embodiments, the devices and methods can use randomized stimulation patterns to prevent the person's brain from becoming accustomed to, and ignoring, the therapeutic stimulation from the tactile stimulation device. In certain embodiments, the devices are positioned against an external surface of a person's skin in a region proximate the lower spine.

In certain embodiments, a method is disclosed for enhancing sleep. The method includes positioning one or more tactile stimulators against an external surface of a person's skin at a location proximate the intersection between lumbar and sacral regions of the person's spinal column. The method further includes actuating the one or more tactile stimulators to produce randomized vibrations in response to an actuation signal.

In other embodiments, an external electromechanical apparatus is disclosed for enhancing sleep. The apparatus includes one or more tactile stimulators configured to be placed against a person's back proximate the lower spine and a controller configured to occasionally actuate the one or more tactile stimulators in a randomized manner. The apparatus also includes a power supply and a power control circuit configured to provide power from the power supply to the one or more tactile stimulators and the controller.

In other embodiments, an external electromechanical tactile stimulation device adapted to be positioned against a person's lower spine is disclosed. The device comprises a belt and an electromechanical stimulus unit coupled to the belt. The electromechanical stimulus unit includes at least two vibrators configured to be centered about the person's lower spine and a controller in communication with the at least two vibrators to control actuation of the at least two vibrators. The electromechanical stimulus unit also includes a battery that provides power for operation of the controller and the at least two vibrators and a switch that selects between a responsive mode and a standalone mode. The controller can be configured to actuate the at least two vibrators in response to control signals received from one or more remote sensors in the responsive mode. The controller can be configured to occasionally actuate the at least two vibrators in a randomized manner in the standalone mode.

In other embodiments, a sleep enhancement system is disclosed. The sleep enhancement system includes a remote control unit and a tactile stimulus unit in communication with the remote control unit. The remote control unit includes a sensory input device configured to detect a sensory input associated with a sleeping person, a signal processing module configured to generate a digital actuation signal when the sensory input exceeds a specified threshold level, and a transmitter configured to transmit the actuation signal. The tactile stimulus unit includes a receiver in communication with the transmitter and configured to receive the actuation signal; at least one tactile stimulator configured to be positioned proximate the sleeping person's lower spine, the at least one tactile stimulator configured to exert pressure on a skin surface adjacent its position; and a controller in communication with the at least one tactile stimulator to control actuation of the at least one tactile stimulator for a predetermined period of time in response to the received actuation signal from the remote control unit, wherein the specified threshold level can be adjustable by a user, and wherein the controller can be configured to output a sequence of output pulses having randomized pulse widths and randomized pause times between successive output pulses for the predetermined period of time.

For purposes of summarizing the disclosure, certain aspects, advantages, and novel features have been described herein. It is to be understood that not necessarily all such advantages may be achieved in accordance with any particular embodiment. Thus, the apparatus, methods, and systems described herein may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 illustrates a functional block diagram of an electromechanical stimulus unit according to certain embodiments.

FIG. 2 illustrates a perspective view of an electromechanical tactile stimulation apparatus placed proximate the lower spine of a user according to certain embodiments.

FIG. 3 illustrates a functional block diagram of a remote control unit usable with the electromechanical stimulus unit of FIG. 1.

### DETAILED DESCRIPTION

Sonic emanations of a sleeper and many other sleep disorders may be caused by uneven muscle tone throughout the body due to the lack of proper interfacing between the brain and the other parts of the body. Many studies indicate that a person's quality and quietness of sleep can be markedly improved by performing an effective method of relaxation just before sleeping. But many people do not have the time to do this, or are “too tired” to relax, especially if any effort is required. Accordingly, disclosed herein are devices and methods for enhancing sleep by improving muscle tone throughout the entire body using an electromechanical tactile stimulation device.

Some embodiments include an electromechanical tactile stimulation device that is compact, inexpensive, safe, and easy to use. In certain embodiments, the device consists of an up to twelve square inch low profile padded, flexible enclosure attached to an elastic belt and worn by a sleeper around his waist with the enclosure touching his skin. Within the enclosure, in certain embodiments, is a battery-powered controller that controls one or more stimulators mounted loosely on a flexible circuit board within small pockets of the flexible enclosure. These stimulators can operate independently of each other, and can be driven in a separate and randomized manner with varying force and at a varying rate to contact the sleeper's skin in such a manner as to resemble the touching by another person's fingertips of one hand. In certain embodiments, the device mildly and quietly influences a person's central nervous system at the lower end of his spinal cord with a randomized stimulus that will minimize its accommodation factor, while not waking the sleeper, nor disturbing others around them. The placement of the device proximate the lower spine of the sleeper can induce the spread of uniform muscle tone more evenly throughout the body.

Certain embodiments of an external electromechanical device induce a quieter, more restful sleep while suppressing the most common sleep disorders, or parasomnias. In some embodiments, the device is triggered by a remote sensing unit, which responds to audible snoring or the excessive movement of the sleeper and activates the electromechanical device worn by the sleeper for a preset short period of time (e.g., a few minutes). In this way, the remote sensing unit does not respond to the ordinary movement of a sleeper, as it would if worn on the person of a sleeper and able to contact the bedding. In other embodiments, the device is configured to operate as a standalone unit with a power switch and a connector to recharge a power supply. The therapeutic vibrations, or oscillations, of the one or more stimulators can be activated for a randomized period of time and then deactivated for a randomized period of time in such a way that minimizes the brain's accommodation to the stimulus.

Although not required to make or use the presently disclosed device, the following provides some scientific basis to the results achieved by embodiments disclosed herein. Mild mechanical stimulation of the Meissner corpuscles and Merkels discs located near the base of the spinal cord enables the reticular formation at the base of the brain. These Meissner corpuscles and Merkels discs are nerve endings, which are known to be responsible for the sensation of touch. The reticular formation consists of a dense neuron-fiber center, which involves millions of neurons at the center of the brain stem. Recent discoveries indicate that millions of bits of information from nerve endings throughout the entire human body are decoded and sorted into order of importance in the reticular formation.

It is also thought that this neuron center controls consciousness and awareness of the human body, along with the body's level of muscle tone. The reticular formation is believed to be the interface control of the brain that blocks the input of insignificant or monotonous data from the myriad of nerve ending sensors for various physiological functions of the body. Some of this data is necessary for the reticular formation to maintain control of a human body's overall muscle tone. In certain embodiments, the electromechanical tactile stimulation device described herein resets this function and allows the brain to properly and continually control uniform muscle tone. In addition to resetting this function, the electromechanical tactile stimulation device can train the brain to function correctly on its own within a few weeks of using this method of stimulus and decreases its necessity.

According to the Biological Law of Uni-Directional Rate Sensitivity, formulated by renowned neuroscientist Dr. Manfred Clynes, the human nervous system is more sensitive to changing stimulation than to constant stimulation. Hence, nerve sensors become desensitized over time through constant stimulation. This law explains the ability of the human brain to adapt to monotonous stimuli and eventually ignore them. In certain embodiments, the devices and methods described herein overcome this biological phenomenon by utilizing randomization.

In certain embodiments, an external electromechanical device is provided that can be easily worn and activated manually by a mode switch when necessary to enable a standalone operating mode that will generally induce a uniform muscle tone while awake or asleep with very little excessive muscle tension anywhere in the body. This standalone mode can be useful in suppressing attacks of asthma and lowering of blood pressure, as it reduces anxiety, one of the common causes of these and other symptoms of stress. In other embodiments, an externally attached electromechanical device configured with a Hall effect breathing sensor is provided that will detect a shallow breathing episode while a person is asleep and remotely activate the electromechanical device in order to suppress an asthma attack before it progresses.

In certain embodiments, the devices and methods can be used to treat various sleep disorders, such as obstructive sleep apnea and central sleep apnea. In other embodiments, the devices and methods can also be used to cure asthma, lower blood pressure, and/or prevent snoring. In still other embodiments, the devices can be programmed to generate basic emotions in a person while they experience Rapid Eye Movement (REM) sleep. For example, Sentic States, as discovered by Manfred Clynes, can be programmed into various embodiments of the devices. In certain embodiments, Sentic States can be used in conjunction with certain embodiments to “orchestrate” the dreams of a sleeper.

The features of the systems and methods will now be described with reference to the drawings summarized above. Throughout the drawings, reference numbers are re-used to indicate correspondence between referenced elements. The drawings, associated descriptions, and specific implementation are provided to illustrate various embodiments and not to limit the scope of the disclosure.

In addition, methods and functions described herein are not limited to any particular sequence, and the acts relating thereto can be performed in other sequences that are appropriate.

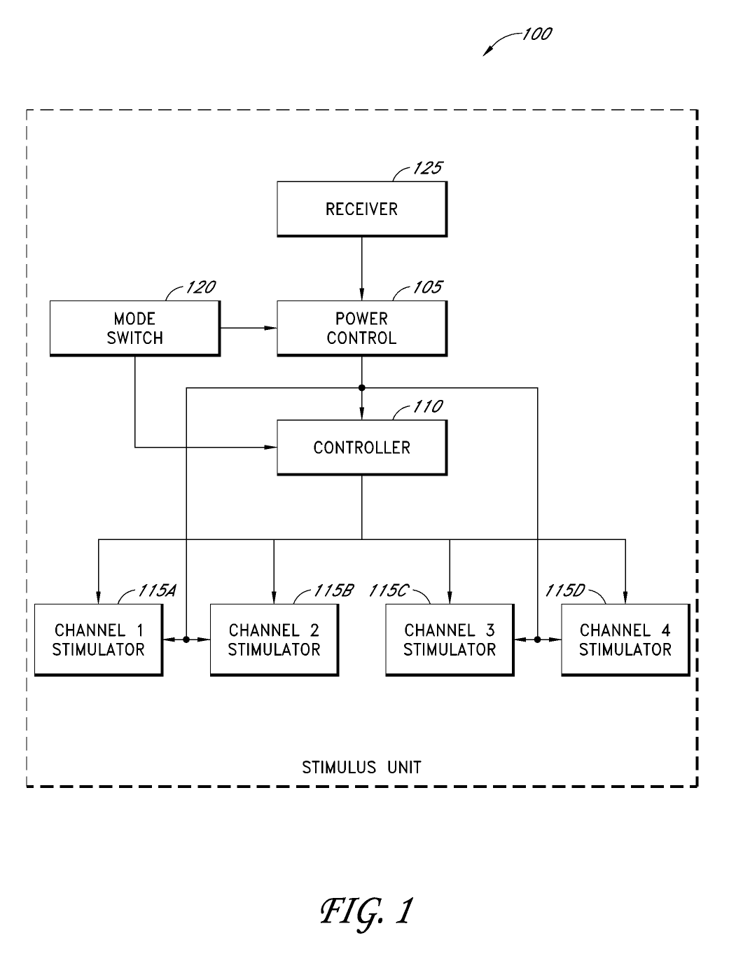

FIG. 1 illustrates a functional block diagram of an electromechanical stimulus unit 100. The stimulus unit 100 comprises a power control module 105, a controller/processor 110, four stimulators 115A-D, a mode switch 120 and a receiver 125.

In certain embodiments, the power control module 105 activates power to the rest of the stimulus unit 100 from a power supply (e.g., a storage battery or a capacitive storage device (not shown)). For example, the power control module 105 can control power supplied to the controller 110, the stimulators 115 and the receiver 125. In some embodiments, operating power is applied continuously to the receiver 125 and the power control module 105 whenever the power supply is activated on the stimulus unit 100. Activation of the stimulus unit 100 can comprise manually pressing a power-on button or toggling a power switch. In some embodiments, the mode switch 120 can be used to activate the power supply. In other embodiments, the power control module 105 is activated upon receipt by the receiver 125 of an actuation signal from a remote sensing and control unit. The power control module 105 can include components to step up or step down power in order to meet the power requirements of the controller 110, the stimulators 115 and the receiver 125. In certain embodiments, the power supply is capable of being recharged via an external connector.

The controller 110 controls the operation of the stimulators 115. The controller 110 comprises a general or a special purpose microprocessor. In certain embodiments, the controller 110 comprises a peripheral interface controller (PIC) chip. In other embodiments, the controller 110 can comprise an application-specific integrated circuit (ASIC) or one or more modules configured to execute on one or more processors.

In certain embodiments, upon power activation of the controller 110, a program stored within the controller 110 outputs a sequence of randomized pulses. The output pulses can energize drivers (not shown) for each respective stimulator channel, which in turn can activate each of the respective stimulators 115. In certain embodiments, the drivers comprise one or more transistors. For example, each driver can comprise a circuit of one or more bipolar junction transistors (BJTs) or field effect transistors (FETs). The length of time that each stimulator 115 is activated corresponds to the pulse-width time of each output pulse. In certain embodiments, the pulse-width time of each pulse varies randomly from 0.1 seconds to 3.3 seconds. In other embodiments, the pulse-width time can vary randomly from 0.05 seconds to 5 seconds. The pause time between each successive pulse of the output sequence can also be varied randomly. In certain embodiments, the pause time varies randomly from 0.5 to 3.3 seconds. In other embodiments, the pause time varies randomly from 0.1 to 10 seconds. In still other embodiments, other suitable pulse-width and pause times can be used.

The stimulators 115 are configured to provide tactile stimulation. In certain embodiments, the stimulators 115 comprise vibrators. In other embodiments, the stimulators 115 comprise solenoids. Although FIG. 1 illustrates four stimulators, any number of stimulators can be used without departing from the spirit and scope of the disclosure. For example, embodiments of the electromechanical stimulation unit 100 can include one or two stimulators.

The mode switch 120 can be configured to alternate operation of the stimulus unit 100 between a standalone mode and a responsive mode. In certain embodiments, the mode switch 120 controls activation of the power control module 105. The mode switch 120 is in electrical communication with the controller 110 and can be configured to switch the controller 110 between operation in the standalone mode and the responsive mode. In certain embodiments, the electromechanical stimulus unit 100 does not include the mode switch 120 and operates in only the standalone mode or the responsive mode. For example, in some embodiments, the electromechanical stimulus unit 100 includes a power switch with an internal power source and only operates in a standalone mode. In certain embodiments, a user interface includes a power switch and a connector to recharge the battery. The user interface can include a battery warning light to notify a user that the battery needs to be recharged. In the standalone mode, the electromechanical stimulus unit 100 can operate on its own, without receipt of any communication from remote, or peripheral, devices. In the responsive mode, the electromechanical stimulus unit 100 can operate in response to actuation signals received from a remote control unit or from individual remote sensors in communication with the electromechanical stimulus unit 100.

In certain embodiments, when the electromechanical stimulus unit 100 is operating in the standalone mode, the mode switch 120 enables direct power to be supplied to the controller 110 via the power control module 105 and also initiates a randomized operating mode preprogrammed in the controller 110. In certain embodiments, the controller 110 generates randomized sequences of output pulses to be sent to the stimulators 115 at a predetermined interval (e.g., every ten minutes, every half hour, every hour, every other hour). In certain embodiments, the predetermined interval can be adjusted by a user. In other embodiments, there is no predetermined interval and the stimulators 115 can be activated for a randomized period of time and then deactivated for a randomized period of time.

As described above, when operating in the standalone mode, the controller 110 can randomly vary the pulse widths and pulse rates of the output pulses sent to each of the stimulator drivers. The controller 110 can also be configured to randomly vary the total duration of each successive randomized sequence of output pulses. In certain embodiments, the total duration of each successive sequence is randomized to vary from three to six minutes. In other embodiments, the total duration of each successive sequence is randomized to vary from one to twenty minutes. In certain embodiments, each stimulator can operate independently of each other, and can be driven in a separate and randomized manner with varying force and at a varying rate.

In embodiments where there is no predetermined interval, the wait time, or disable period, between successive sequences can also be randomized, thereby further preventing the brain from becoming accustomed to, and/or ignoring, the therapeutic vibrations. In certain embodiments, the disable period is randomized to vary from seven to eleven minutes. In other embodiments, the disable period between successive sequences is randomized to vary from five to thirty minutes. In still other embodiments, the disable period can be up to several hours.

The receiver 125 is configured to receive transmission signals from one or more sensory input devices monitoring various behavioral aspects of a sleeping person. In certain embodiments, the receiver 125 comprises a Frequency Modulation (FM) receiver and an FM receiving antenna. In certain embodiments the output of the receiver 125 is used to activate the power control module 105 operating in the responsive mode.

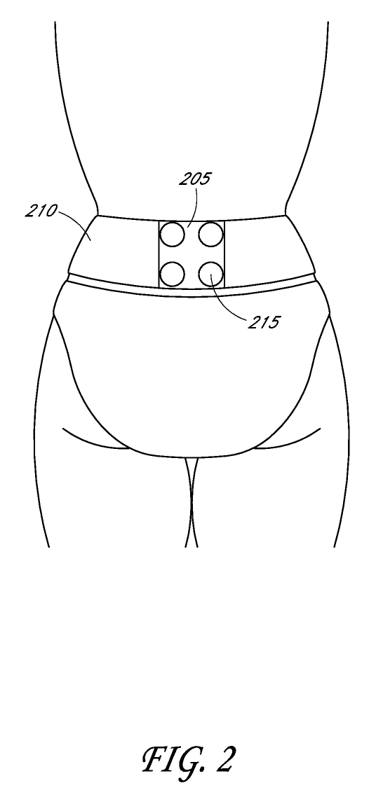

FIG. 2 illustrates a perspective view of an electromechanical tactile stimulation apparatus 200, according to certain embodiments. The tactile stimulation apparatus 200 includes an enclosure 205 attached to a belt 210. As shown, the belt 210 is configured to be worn proximate the waist region of the wearer. The belt 210 can be attached around the waist region of the wearer by any suitable fixation means (e.g., Velcro, a clip, a latch). In certain embodiments the belt 210 is configured to be elastic. The enclosure 205 can be fixedly attached or removably attached to the belt 210 in various embodiments. For example, the enclosure 205 can be secured to the belt 210 by means of Velcro or other suitable attachment means.

As illustrated in FIG. 2, the enclosure 205 is positioned in the small of the wearer's back proximate the lower spine. In certain embodiments, the enclosure 205 is positioned at a point between the lumbar and sacral regions of the spinal column. In other embodiments, the enclosure 205 can be positioned on the abdominal area of the wearer at a location coinciding with the illustrated location in FIG. 2. In yet other embodiments, the enclosure 205 can be positioned at other locations on the wearer's body. In certain embodiments, the enclosure 205 can comprise various forms of padding or cushioning to enhance comfort when the wearer is sleeping on his back. In other embodiments, the enclosure 205 is formed of a flexible material.

In certain embodiments, the electromechanical stimulus unit 100 can be inserted into a molded pouch (not shown) of the enclosure 205. For example, the electromechanical stimulus unit 100 can be mounted on a circuit board sized and configured to fit within the molded pouch. In some embodiments, the electromechanical stimulus unit 100 is retained within the pouch by means of Velcro, zipper or the like. The stimulators 115A-D can each be attached to one corner of the circuit board so as to fit in a molded pocket 215 of the enclosure 205 in such a way as to allow sufficient range of motion to vibrate, or oscillate, freely. The stimulators 115 are advantageously centered and arranged evenly about the desired location, regardless of the number of stimulators used. In certain embodiments, the circuit board comprises a flexible circuit board.

As shown in FIG. 1, each of the components of the electromechanical stimulus unit 100 can be mounted on a single integrated circuit board. In other embodiments, one or more of the components (e.g., the vibrators 115 or the power supply) can be connected via electrical or optical cables to the controller 110.

In other embodiments, any other suitable device capable of producing a vibration or other tactile stimulation can be inserted within the flexible enclosure 205. For example, a cellular phone, a Personal Digital Assistant (PDA), or other handheld device equipped with one or more vibrators can be programmed to operate in a standalone mode as described above and inserted into the flexible enclosure 205 and/or attached to the belt 210.

In certain embodiments, the electromechanical stimulus unit 100 is mounted directly to the belt and not within a pouch. For example, the electromechanical stimulus unit 100 can be coupled to the belt via a clip, industrial-grade Velcro, adhesive, and the like. In other embodiments, the electromechanical stimulus unit 100 can be held in place without the use of a belt. For example, the electromechanical stimulus unit 100 can be removably attached to a waist band of a person's pants, shorts, or undergarments (e.g., via a clip, latch, or Velcro).

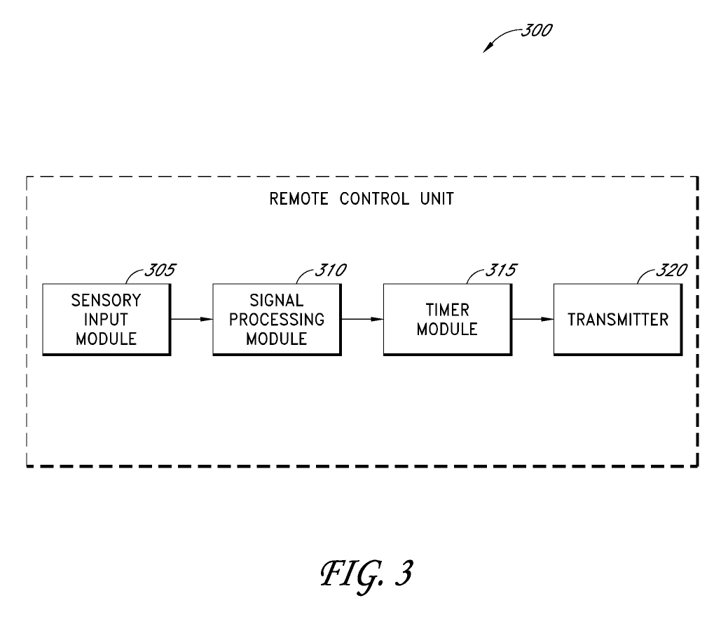

FIG. 3 illustrates a functional block diagram of a remote control unit 300, according to certain embodiments. The remote control unit 300 can be used, for example, in conjunction with the electromechanical stimulus unit 100 when the electromechanical stimulus unit 100 is operating in the responsive mode. The remote control unit 300 includes one or more sensory input devices 305, a signal processing module 310, a timer module 315, and a transmitter 320.

The sensory input devices 305 can include a microphone, a motion detector, a breathing sensor, a pressure sensor, and/or the like. The sensory input devices 305 are configured to monitor various behavioral and physiological conditions of a sleeping person. For example, a microphone can be configured to detect the sonic emanations, or snoring sounds, of a sleeper and a motion detector can be configured to detect motion of the sleeper's body while sleeping. A breathing sensor can be configured to detect variations in breathing, such as an initial episode of shallow breathing signaling an oncoming attack of asthma. A pressure sensor can be mounted on the sleeper's back and can be configured to detect when the sleeper is on his back. In certain embodiments, the pressure sensor can generate a signal to automatically increase the intensity of the therapeutic vibrations provided by the electromechanical stimulus unit 100 when the pressure sensor detects that the sleeper is on his back. The increased intensity can advantageously be used to account for any extra pressure dampening the vibrations.

The signal processing module 310 is configured to receive electrical signals from the sensory input devices 305 and to determine whether or not to generate a control signal to transmit to the electromechanical stimulus unit 100. The signal processing module 310 can include a microphone preamp circuit, an audio level detection circuit, a buffer, one or more filters, an analog-to-digital converter, and/or other signal processing or data storage devices. The signal processing module 310 can also include one or more potentiometers configured to allow various threshold sensitivity levels to be adjusted.

As an example, the signal processing module 310 can be configured to receive an analog signal from a microphone. The analog signal is then amplified by a microphone preamp circuit and then processed by an audio level detection circuit to produce a digital on-or-off output to the timer module 315. External adjustment of at least one internal potentiometer can be used to set the sensitivity of the audio level detection circuit.

As another example, the signal processing module 310 can be configured to receive a digital on-or-off signal from a motion detector. External adjustment of another potentiometer can be used to set the degree of motion required to generate an output signal of the motion detector. The signal processing module 310 can buffer the motion detector output signal before feeding it to the timer module 315.

The timer module 315 can be configured to control the amount of time that the transmitter 320 is transmitting. The timer module 315 can also provide a delay such that transmission only occurs when a sensor is triggered over a prolonged period of time (e.g., a few minutes). In certain embodiments, external adjustment of an internal potentiometer within the timer module 315 is used to set the time period required to elapse between the switching on and the switching off of the transmitter 320 over a range of from one to ten minutes. In other embodiments the range can be from twenty seconds to thirty minutes.

The transmitter 320 is configured to transmit actuation signals to the electromechanical stimulus unit 100. When switched on, the transmitter 320 emits a low power transmission signal via a transmission antenna to the receiving antenna of the receiver 125. In certain embodiments, the transmission signal frequency is within the range of FCC approved radio frequencies for such remote control devices.

Certain embodiments provide a remote controlled, four-channel electromechanical stimulation device designed to drive the four small vibrators in such a manner as to simulate the touching of finger tips to the lower back of the sleeper or anyone wishing to induce a uniform muscle tone within themselves. Some embodiments provide circuitry which produces randomized pulses that drive vibrators in short bursts which accomplish this simulated touching, as if the finger tips of another person's hand are dancing lightly at a location on the subject's lower back that is level with the base of their spinal cord. Use of the electromechanical tactile stimulation device according to certain embodiments is advantageous, as this location is not reachable by another person when a sleeper reclines on their back.

In an alternate embodiment (not shown) of the presently disclosed device, a commercially available chest-mounted Hall effect breathing sensor (not shown) with an analog signal output is connected to an input of the remote control unit 300 which is attached to the chest-mounted breathing sensor. The transmitter 320 can be replaced by a processor, such as Microchip Corporation's rfPIC12F675. The processor can be a programmable interface microcontroller capable of being programmed to detect variations in breathing rate represented by the analog signal produced by the chest-mounted Hall effect breathing sensor and then to transmit an FM signal to trigger the stimulus unit 100 via the receiver 125. In this embodiment the output of the chest-mounted breathing sensor is connected directly to an input of the processor and the processor is programmed to transmit for a time period that is adjusted by a potentiometer connected directly to the processor (e.g., rfPIC chip). The transmission time period can be programmed to be between one and ten minutes. In other embodiments, the transmission time period can be programmed to be between twenty seconds and thirty minutes. The alternate embodiment of the presently disclosed device configured as described above can be used for detecting variations in breathing such as an initial episode of shallow breathing signaling an oncoming attack of asthma.

In certain embodiments, the electromechanical stimulus unit 100 can be configured to provide adjustable intensity (e.g., increase/decrease the intensity of the vibrations) via an external switch or knob. In other embodiments, the electromechanical stimulus unit 100 can be configured to allow a user to adjust the pulse width ranges generated by the controller 110 via an external switch or knob. In still other embodiments, the electromechanical stimulus unit 100 includes a display. For example, the display can be similar to an LCD display on a PDA or cell phone. The display can be large enough to include a multi-character message, a multi-line message and/or graphical user interface (GUI) display objects. For example, the display can be used to display the number of times the electromechanical stimulus unit 100 was activated over a specified period of time, the battery level or battery charging status, the intensity level, and/or the connection status between the electromechanical stimulus unit 100 and any remote sensors. The display can be a touchscreen display such that the display operates as an input device to the electromechanical stimulus unit 100. For example, the display can be used to adjust the pulse-width range, change the intensity of the therapeutic vibrations, or switch between the responsive and standalone modes. In certain embodiments, the touchscreen display can replace the need for additional external buttons or switches.

In other embodiments, the devices and methods described above can be used in conjunction with erotic vibrators. For example, the randomized stimulations described above can be incorporated into an erotic vibrator such that a person would not become accustomed to the stimulus. It should be appreciated that an erotic device incorporating the random vibrations described above could also be used for enhancing sleep.

In still other embodiments, the devices and methods described above can be used in conjunction with portable consumer electronic devices, such as a cellular phone, a PDA, a smartphone, and the like. In certain embodiments the existing vibrator within a portable consumer electronic device can be programmed to operate in a standalone mode with randomized vibrations as described herein.

While certain embodiments have been described, these embodiments have been presented by way of example only, and are not intended to limit the scope of the disclosure. Indeed, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

What is claimed is:

1. An external electromechanical tactile stimulation device adapted to be positioned against a person's lower spine, the device comprising:
a belt; and
an electromechanical stimulus unit coupled to the belt, the electromechanical stimulus unit comprising:
at least two vibrators configured to be centered about a person's lower spine;
a controller in communication with the at least two vibrators to control actuation of the at least two vibrators;
a battery that provides power for operation of the controller and the at least two vibrators; and
a switch that selects between a responsive mode and a standalone mode;
wherein, in the responsive mode, the controller is configured to actuate the at least two vibrators in response to control signals received from one or more remote sensors; and
wherein, in the standalone mode, the controller is configured to occasionally actuate the at least two vibrators in a randomized manner.

2. The tactile stimulation device of claim 1, wherein the electromechanical stimulus unit is inserted within a pocket of a flexible enclosure coupled to the belt.

3. The tactile stimulation device of claim 1, wherein the electromechanical stimulus unit is removably attached to the belt.

4. The tactile stimulation device of claim 1, further comprising a pressure sensor in communication with the controller, wherein the controller adjusts an intensity level of the at least two vibrators based on the detected pressure.

5. The tactile stimulation device of claim 1, wherein the electromechanical stimulus unit is mounted on a flexible circuit board.

6. The tactile stimulation device of claim 1, further comprising a receiver for receiving actuation signals from a remote sensory input device when the controller is operating in the responsive mode.

7. The tactile stimulation device of claim 1, wherein the controller comprises stored instructions that, upon being executed thereon, output a sequence of randomized output pulses having randomized pulse widths and randomized wait times between successive output pulses.

8. The tactile stimulation device of claim 7, wherein a range of the randomized pulse widths is adjustable.

9. A sleep enhancement system, the system comprising:
a remote control unit, the remote control unit comprising:
a sensory input device configured to detect a sensory input associated with a sleeping person;
a signal processing module configured to generate a digital actuation signal when the sensory input exceeds a specified threshold level;
a transmitter configured to transmit the actuation signal; and
a tactile stimulus unit in communication with the remote control unit the tactile stimulus unit comprising:
a receiver in communication with the transmitter and configured to receive the actuation signal;
at least one tactile stimulator configured to be positioned proximate the sleeping person's lower spine, the at least one tactile stimulator configured to exert pressure on a skin surface adjacent its position; and
a controller in communication with the at least one tactile stimulator to control actuation of the at least one tactile stimulator for a predetermined period of time in response to the received actuation signal from the remote control unit;
wherein the specified threshold level is adjustable by a user;
wherein the controller is configured to output a sequence of output pulses having randomized pulse widths and randomized pause times between successive output pulses for the predetermined period of time.

10. The sleep enhancement system of claim 9, wherein the at least one tactile stimulator is a vibrator.

11. The sleep enhancement system of claim 9, wherein a range of the randomized pulse widths is adjustable.

12. The sleep enhancement system of claim 9, wherein an intensity level of the at least one tactile stimulator is adjustable.

13. The sleep enhancement system of claim 9, wherein the sensory input device is a microphone or a motion detector.

14. The sleep enhancement system of claim 9, wherein the sensory input device is a pressure sensor.

15. A method of enhancing sleep, the method comprising:
positioning one or more tactile stimulators against a person's skin at a location proximate the intersection between lumbar and sacral regions of the person's spinal column;
actuating the one or more tactile stimulators to produce randomized vibrations in response to an actuation signal.

16. The method of claim 15, wherein the actuation signal comprises a randomized sequence of pulses generated by a programmable controller in communication with the one or more tactile stimulators, each pulse having a randomized pulse width.

17. The method of claim 15, further comprising detecting pressure exerted between the person's skin and a contact surface via a pressure transducer.

18. The method of claim 17, further comprising automatically adjusting the intensity of the vibrations in response to the detected pressure.

19. An external electromechanical apparatus for enhancing sleep, the apparatus comprising:
one or more tactile stimulators configured to be placed against a person's back proximate the lower spine;
a controller configured to occasionally actuate the one or more tactile stimulators in a random manner;
a power supply; and
a power control circuit configured to provide power from the power supply to the one or more tactile stimulators and the controller.
